(19) United States
(12) Patent Application Publication
(10) Pub. No.: US 2025/0352812 A1
(43) Pub. Date: Nov. 20, 2025

(54) IMPLANTABLE MEDICAL LEAD IMPLANT TOOL

(71) Applicant: Medtronic, Inc., Minneapolis, MN (US)

(72) Inventors: Matthew J. Hoffman, St. Paul, MN (US); William J. Clemens, Fridley, MN (US); Linnea R. Lentz, Stacy, MN (US); Noelle C. Neafus, St. Paul, MN (US)

(21) Appl. No.: 18/871,713

(22) PCT Filed: Jun. 16, 2023
(86) PCT No.: PCT/IB2023/056266

§ 371 Date: Dec. 4, 2024

Related U.S. Application Data
(60) Provisional application No. 63/366,743, filed on Jun. 21, 2022.

(51) Int. Cl. A61N 1/39; A61N 1/05

(52) U.S. Cl. CPC A61N 1/39622; A61N 1/0504; A61N 1/0563

(57) ABSTRACT

A medical device system includes an implantable medical lead including a lead body defining a proximal end and a distal portion, and at least a part of the distal portion of the lead body defines an undulating configuration. The medical device system also includes an introducer tool defining a lumen configured to receive the distal portion, and the introducer tool is configured to limit rotation of the distal portion within the lumen.

This application claims the benefit of U.S. Provisional Patent Application Ser. No. 63/366,743, filed 21 Jun. 2022, the entire content of which is incorporated herein by reference.

### TECHNICAL FIELD

The present application relates to implantable medical leads and, more particularly, techniques for implanting implantable medical leads.

### BACKGROUND

Malignant tachyarrhythmia, for example, ventricular fibrillation (VF), is an uncoordinated contraction of the cardiac muscle of the ventricles in the heart, and is the most commonly identified arrhythmia in cardiac arrest patients. If this arrhythmia continues for more than a few seconds, it may result in cardiogenic shock and cessation of effective blood circulation. As a consequence, sudden cardiac death (SCD) may result in a matter of minutes.

In patients with a high risk of VF, the use of implantable systems, such as an implantable cardioverter defibrillator (ICD) system, has been shown to be beneficial at preventing SCD. Implantable systems, such as pacemakers with or without cardioversion or defibrillation capabilities, may also treat other cardiac dysfunction, such as bradycardia and heart failure. Such implantable systems may include electrical devices configured to deliver therapy via electrodes. Therapy may include shocks and/or anti-tachycardia pacing (ATP). The implantable systems may also be configured to deliver cardiac pacing to, for example, treat bradyarrhythmia or for cardiac resynchronization therapy (CRT).

An implantable system may include one or more implantable medical leads. A distal portion of an implantable medical lead may include one or more electrodes, and may be positioned at a target location within the patient for delivery of electrical therapy and/or electrical sensing via the electrodes. A proximal end of the lead may be coupled to the implantable system. The implantable system may also include one or more housing electrodes, which are sometimes referred to as can electrodes, for delivery of therapy and/or sensing.

Owing to the inherent surgical risks in attaching and replacing implantable medical leads directly within or on the heart, subcutaneous implantable systems have been devised, in which the implantable system and leads are located subcutaneously outside of the thorax. It has also been proposed that the distal portion of a lead of an implantable system may be implanted within the thorax, but not in contact with the heart, e.g., substernally. Additionally, it has been proposed to implant the distal portion of a lead of an implantable system within an extracardiac vessel that is within the thorax, such as the internal thoracic vein (ITV), the intercostal veins, the superior epigastric vein, or the azygos, hemiazygos, and accessory hemiazygos veins.

Implantable medical leads are also used to deliver therapies to tissues other than the heart. Implantable medical leads may be used to position one or more electrodes within or near target nerves, muscles, or organs to deliver electrical stimulation to such tissues. As examples, implantable medical leads may be positioned in the epidural space to deliver spinal cord stimulation, or proximate to other nerves, such as pelvic nerves or renal nerves, to deliver neurostimulation to the nerves.

### SUMMARY

Relative to electrodes on or within the heart, delivery of pacing pulses or defibrillation using electrodes of extravascular or other extracardiac leads may require higher energy levels to capture and/or defibrillate the heart. The shape and/or positioning of the electrodes, or a lead body on which the electrodes are placed, may be chosen to improve delivery of energy to the heart while avoiding and/or reducing energy delivered to extracardiac tissue. The lead body and/or electrodes of extravascular implantable cardioverter defibrillator (EV-ICD) having an undulating shape, a serpentine shape, or the like, may “flip” when deploying the lead, e.g., with the electrodes and/or coils facing the opposite direction than intended during implantation. For example, an EV-ICD may be intended to be implanted with defibrillation electrodes oriented towards the patient's right side, and pacing electrodes towards the patient's left side, and the EV-ICD lead may “corkscrew” while being advanced within a lumen of a conventional introducer tool so as to “flip” when deployed, e.g., with the defibrillation electrodes oriented towards the patient's left side, and pacing electrodes towards the patient's right side.

This disclosure describes implantable medical leads and implantable systems, such as ICD systems, utilizing the leads. More particularly, this disclosure describes systems including an introducer tool configured to limit rotation of a lead during implantation. For example, a catheter may have a distal portion defining a lumen having a noncircular shape, e.g., rectangular, square, elliptical, or the like, configured to “lock” a portion of the lead into a preferential rotational orientation and allow a clinician to visually determine the orientation of the lead via imaging (e.g., fluoroscopy), prior to implant.

In one example, this disclosure describes a medical device system including: an implantable medical lead includes a lead body defining a proximal end and a distal portion, wherein at least a part of the distal portion of the lead body defines an undulating configuration; and an introducer tool defining a lumen configured to receive the distal portion, wherein the introducer tool is configured to limit rotation of the distal portion within the lumen.

In another example, this disclosure describes a method including: positioning an introducer tool at an implant location within a patient, wherein the introducer tool defines a lumen configured to receive a distal portion of an implantable medical lead, wherein the introducer tool is configured to limit rotation of the distal portion within the lumen; inserting the distal portion of the implantable medical lead into the lumen, wherein the implantable medical lead comprises a lead body defining a proximal end and the distal portion, wherein at least a part of the distal portion of the lead body defines an undulating configuration; advancing, through the lumen, the distal portion to the implant location; and deploying the distal portion to be in the undulating configuration at the implant location and with a predetermined rotational orientation.

In another example, this disclosure describes a medical device system including: an implantable medical lead comprising a lead body defining a proximal end and a distal portion, wherein at least a part of the distal portion of the lead body defines an undulating configuration; and an introducer tool defining a non-circular lumen configured to receive the undulating configuration and to lock the undulating configuration to a particular rotational orientation within the lumen.

This summary is intended to provide an overview of the subject matter described in this disclosure. It is not intended to provide an exclusive or exhaustive explanation of the systems, devices, and methods described in detail within the accompanying drawings and description below. Further details of one or more examples are set forth in the accompanying drawings and the description below. Other features, objects, and advantages will be apparent from the description and drawings, claims, and from the statements provided below.

### BRIEF DESCRIPTION OF DRAWINGS

FIG. 1A is a front view of a patient with an extravascular ICD system including a lead implanted intra-thoracically.

FIG. 1B is a side view of the patient with the extravascular ICD system having the lead implanted intra-thoracically.

FIG. 1C is a transverse view of the patient with the extravascular ICD system having the lead implanted intra-thoracically.

FIG. 2 is a conceptual diagram illustrating a distal portion of an example implantable medical lead deployed in a first rotational orientation.

FIG. 3 is a conceptual diagram illustrating the distal portion of the example implantable medical lead of FIG. 2 deployed in a second rotational orientation.

FIG. 4 is a conceptual, cross-sectional view of a distal portion of an example implantable medical lead within a lumen of an example introducer tool.

FIG. 5 is a conceptual, cross-sectional view of a distal portion of an example implantable medical lead within a lumen of another example introducer tool.

FIG. 6 is a conceptual, cross-sectional view of an example dilator tool within a lumen of another example introducer tool.

FIG. 7 is a flow diagram illustrating an example technique for implanting an implantable medical lead via an example introducer tool.

FIG. 8 is a functional block diagram of an example configuration of electronic components of an example ICD.

### DETAILED DESCRIPTION

As used herein, relational terms, such as “first” and “second,” “over” and “under,” “front” and “rear,” and the like, may be used solely to distinguish one entity or element from another entity or element without necessarily requiring or implying any physical or logical relationship or order between such entities or elements.

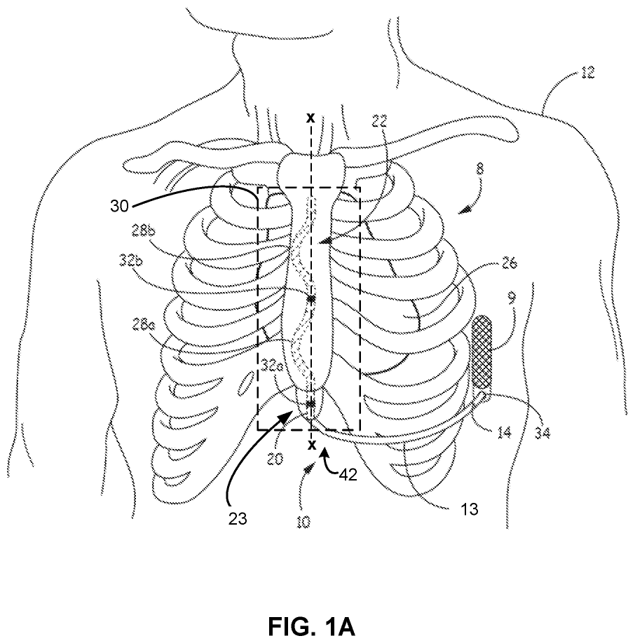

Referring now to the drawings in which like reference designators refer to like elements, there is shown in FIGS. 1A-1C conceptual diagrams illustrating various views of an example extravascular implantable cardioverter-defibrillator (ICD) system 8. ICD system 8 includes an ICD 9 connected to an implantable medical lead 10 and a separately implantable shield 30. FIG. 1A is a front view of a patient 12 implanted with extravascular ICD system 8. FIG. 1B is a side view of the patient 12 implanted with extravascular ICD system 8. FIG. 1C is a transverse view of the patient 12 implanted with extravascular ICD system 8.

ICD 9 may include a housing that forms a hermetic seal that protects components of the ICD 9. The housing of ICD 9 may be formed of a conductive material, such as titanium or titanium alloy, which may function as a housing electrode (sometimes referred to as a can electrode). In some embodiments, ICD 9 may be formed to have or may include a plurality of electrodes on the housing. ICD 9 may also include a connector assembly (also referred to as a connector block or header) that includes electrical feedthroughs through which electrical connections are made between conductors of lead 10 and electronic components included within the housing of ICD 9. As will be described in further detail herein, the housing may house one or more processors, memories, transmitters, receivers, sensors, sensing circuitry, therapy circuitry, power sources and other appropriate components. The housing is configured to be implanted in a patient, such patient 12.

In the examples shown, ICD 9 is implanted extra-thoracically on the left side of the patient, e.g., under the skin and outside the ribcage (subcutaneously or submuscularly). ICD 9 may, in some instances, be implanted between the left posterior axillary line and the left anterior axillary line of the patient. ICD 9 may, however, be implanted at other extra-thoracic locations on the patient as described later.

Lead 10 may include an elongated lead body 13 having a distal portion 16 sized to be implanted in an extravascular location proximate the heart, e.g., intra-thoracically, as illustrated in FIGS. 1A-C, or extra-thoracically. For example, lead 10 may extend extra-thoracically under the skin and outside the ribcage (e.g., subcutaneously or submuscularly) from ICD 9 toward the center of the torso of the patient, for example, toward the xiphoid process 23 of the patient. At a position proximate xiphoid process 23, the lead body 13 may bend or otherwise turn and extend superiorly. The bend may be pre-formed and/or lead body 13 may be flexible to facilitate bending. In the example illustrated in FIGS. 1A-1C, the lead body 13 extends superiorly intra-thoracically underneath the sternum, in a direction substantially parallel to the sternum.

In one example, distal portion 16 of lead 10 may reside in a substernal location such that distal portion 16 of lead 10 extends superior along the posterior side of the sternum substantially within the anterior mediastinum 36 as shown in FIG. 1C. Anterior mediastinum 36 may be viewed as being bounded laterally by pleurae 39, posteriorly by pericardium 38, and anteriorly by the sternum 22. In some instances, the anterior wall of anterior mediastinum 36 may also be formed by the transversus thoracis and one or more costal cartilages. Anterior mediastinum 36 includes a quantity of loose connective tissue (such as areolar tissue), adipose tissue, some lymph vessels, lymph glands, substernal musculature (e.g., transverse thoracic muscle), the thymus gland, branches of the internal thoracic artery, and the ITV.

In another example, lead body 13 may extend superiorly extra-thoracically (instead of intra-thoracically), e.g., either subcutaneously or submuscularly above the ribcage/sternum. Lead 10 may be implanted at other locations, such as over the sternum, offset to the right of the sternum, angled lateral from the proximal or distal end of the sternum, or the like. In other examples, lead 10 may be implanted within an extracardiac vessel within the thorax, such as the ITV, the intercostal veins, the superior epigastric vein, or the azygos, hemiazygos, and accessory hemiazygos veins. In some examples, distal portion 16 of lead 10 may be oriented differently than is illustrated in FIGS. 1A-1C, such as orthogonal or otherwise transverse to sternum 22 and/or inferior to heart 26. In such examples, distal portion 16 of lead 10 may be at least partially within anterior mediastinum 36.

Lead body 13 may have a generally tubular or cylindrical shape and may define a diameter of approximately 3-9 French (Fr). However, lead bodies of less than 3 Fr and more than 9 Fr may also be utilized. In another configuration, lead body 13 may have a flat, ribbon, or paddle shape with solid, woven filament, or metal mesh structure, along at least a portion of the length of the lead body 13. In such an example, the width across lead body 13 may be between 1-3.5 mm. Other lead body designs may be used without departing from the scope of this application.

Lead body 13 may be formed from a non-conductive material, including silicone, polyurethane, fluoropolymers, mixtures thereof, and other appropriate materials, and shaped to form one or more lumens (not shown), however, the techniques are not limited to such constructions. Distal portion 16 may be fabricated to be biased in a desired configuration, or alternatively, may be manipulated by the user into the desired configuration. For example, the distal portion 16 may be composed of a malleable material such that the user can manipulate the distal portion into a desired configuration where it remains until manipulated to a different configuration.

Lead body 13 may include a proximal end 14 and a distal portion 16 which include electrodes configured to deliver electrical energy to the heart or sense electrical signals of the heart. Distal portion 16 may be anchored to a desired position within the patient, for example, substernally or subcutaneously by, for example, suturing distal portion 16 to the patient's musculature, tissue, or bone at the xiphoid process entry site. In some examples, distal portion 16 may be anchored to the patient or through the use of rigid tines, prongs, barbs, clips, screws, and/or other projecting elements or flanges, disks, pliant tines, flaps, porous structures such as a mesh-like elements and metallic or non-metallic scaffolds that facilitate tissue growth for engagement, bio-adhesive surfaces, and/or any other non-piercing elements.

Lead body 13 may define a substantially linear portion 20 (FIG. 1A) as it curves or bends near the xiphoid process 23 and extends superiorly. As shown in FIG. 1A, at least a part of distal portion 16 may define an undulating configuration distal to the substantially linear portion 20. In particular, distal portion 16 may define an undulating pattern, e.g., zig-zag, meandering, sinusoidal, serpentine, or other pattern, as it extends toward the distal end of lead 10. In other configurations, lead body 13 may not have a substantially linear portion 20 as it extends superiorly, but instead the undulating configuration may begin immediately after the bend.

Distal portion 16 includes one or more defibrillation electrodes configured to deliver an anti-tachyarrhythmia, e.g., cardioversion/defibrillation, shock to heart 26 of patient 12. In some examples, distal portion 16 includes a plurality of defibrillation electrodes spaced a distance apart from each other along the length of distal portion 16. In the example illustrated by FIGS. 1A-1C, distal portion 16 includes two defibrillation electrodes 28a and 28b (collectively, “defibrillation electrodes 28”).

Defibrillation electrodes 28 may be disposed around or within the lead body 13 of the distal portion 16, or alternatively, may be embedded within the wall of the lead body 13. In one configuration, defibrillation electrodes 28 may be coil electrodes formed by a conductor. The conductor may be formed of one or more conductive polymers, ceramics, metal-polymer composites, semiconductors, metals or metal alloys, including but not limited to, one of a combination of the platinum, tantalum, titanium, niobium, zirconium, ruthenium, indium, gold, palladium, iron, zinc, silver, nickel, aluminum, molybdenum, stainless steel, MP35N, carbon, copper, polyaniline, polypyrrole and other polymers. In another configuration, each of defibrillation electrodes 28 may be a flat ribbon electrode, a paddle electrode, a braided or woven electrode, a mesh electrode, a directional electrode, a patch electrode or another type of electrode configured to deliver a cardioversion/defibrillation shock to heart 26 of patient 12.

In one configuration, defibrillation electrodes 28 are spaced approximately 0.25-4.5 cm, and in some instances between 1-3 cm apart from each other. In another configuration, defibrillation electrodes 28 are spaced approximately 0.25-1.5 cm apart from each other. In a further configuration, defibrillation electrodes 28 are spaced approximately 1.5-4.5 cm apart from each other.

In the configuration shown in FIGS. 1A-IC, defibrillation electrodes 28 span a substantial part of distal portion 16. Each of defibrillation electrodes 28 may be between approximately 1-10 cm in length, between approximately 2-6 cm in length, or between approximately 3-5 cm in length. However, lengths of greater than 10 cm and less than 1 cm may be utilized in accordance with the techniques of this disclosure. A total length of defibrillation electrode on distal portion 16, e.g., length of the two defibrillation electrodes 28 combined, may vary depending on a number of variables. In one example, the total length may be between approximately 5-10 cm. However, the defibrillation electrodes 28 may have a total length less than 5 cm and greater than 10 cm in other embodiments. In some instances, defibrillation electrodes 28 may be approximately the same length or, alternatively, different lengths.

Defibrillation electrodes 28 may be electrically connected to one or more conductors, which may be disposed in the body wall of lead body 13 or in one or more insulated lumens (not shown) defined by lead body 13. In an example configuration, each of defibrillation electrodes 28 is connected to a common conductor such that a voltage may be applied simultaneously to all defibrillation electrodes 28 to deliver an anti-tachyarrhythmia shock to heart 26. In other configurations, defibrillation electrodes 28 may be attached to separate conductors such that each defibrillation electrode 28 may apply a voltage independent of the other defibrillation electrodes 28. In this case, ICD 9 or lead 10 may include one or more switches or other mechanisms to electrically connect the defibrillation electrodes together to function as a common polarity electrode such that a voltage may be applied simultaneously to all defibrillation electrodes 28 in addition to being able to independently apply a voltage.

Distal portion 16 may also include one or more pacing and/or sensing electrodes configured to deliver pacing pulses to heart 26 and/or sense electrical activity of heart 26. Such electrodes may be referred to as pacing electrodes, sensing electrodes, or pace/sense electrodes. In the example illustrated by FIGS. 1A-IC, distal portion 16 includes two pace/sense electrodes 32a and 32b (collectively, “pace/sense electrodes 32”).

In the illustrated example, pace/sense electrode 32b is positioned between defibrillation electrodes 28, e.g., within a gap between the defibrillation electrodes, and pace/sense electrode 32a is positioned more proximal along distal portion 16 than proximal defibrillation electrode 28a. In some examples, more than one electrode 32 may exist within the gap between defibrillation electrodes 28. In some examples, an electrode 32 is additionally or alternatively located distal of the distalmost defibrillation electrode 28b.

In one example, the distance between the closest defibrillation electrode 28 and electrodes 32 is greater than or equal to approximately 2 mm and less than or equal to approximately 1.5 cm. In another example, electrodes 32 may be spaced apart from the closest one of defibrillation electrodes 28 by greater than or equal to 5 mm and less than or equal to 1 cm. In a further example, electrodes 32 may be spaced apart from the closest one of defibrillation electrodes 28 by greater than or equal to 6 mm and less than or equal to 8 mm.

Electrodes 32 may be configured to deliver low-voltage electrical pulses to the heart or may sense a cardiac electrical activity, e.g., depolarization and repolarization of the heart. As such, electrodes 32 may be referred to herein as pace/sense electrodes 32. In one configuration, electrodes 32 are ring electrodes. However, in other configurations electrodes 32 may be any of a number of different types of electrodes, including ring electrodes, short coil electrodes, paddle electrodes, hemispherical electrodes, or directional electrodes. Each of electrodes 32 may be the same or different types of electrodes as others of electrodes 32. Electrodes 32 may be electrically isolated from an adjacent defibrillation electrode 28 by including an electrically insulating layer of material between electrodes 32 and adjacent defibrillation electrodes 28. Each electrode 32 may have its own separate conductor such that a voltage may be applied to or sensed via each electrode independently from another electrode 32.

Electrodes 28 are referred to as defibrillation electrodes, and electrodes 32 are referred to as pace/sense electrodes, because they may have different physical structures enabling different functionality. Defibrillation electrodes 28 may be larger, e.g., have greater surface area, than pace/sense electrodes 32 and, consequently, may be configured to deliver anti-tachyarrhythmia shocks that have relatively higher voltages than pacing pulses. The relatively smaller size of pace/sense electrodes 32 may provide advantages over defibrillation electrodes for delivering pacing pulses and sensing intrinsic cardiac activity, e.g., lower pacing capture thresholds and/or better sensed signal quality. Nevertheless, a defibrillation electrode 28 may be used to deliver pacing pulses and/or sense electrical activity of the heart, such as in combination with a pace/sense electrode 32.

In the configuration shown in FIGS. 1A-IC, each electrode 32 is substantially aligned along a major longitudinal axis (“x”). In one example, the major longitudinal axis is defined by a portion of elongate body 13, e.g., substantially linear portion 20. In another example, the major longitudinal axis is defined relative to the body of the patient, e.g., along the anterior median line (or midsternal line), one of the sternal lines (or lateral sternal lines), left parasternal line, or other line.

In one configuration, the midpoint of each electrode 32a and 32b is along the major longitudinal axis “x,” such that each electrode 32a and 32b is at least disposed at substantially the same horizontal position when the distal portion is implanted within the patient. In some examples, the longitudinal axis “x” may correspond to a caudal-cranial axis of the patient and a horizontal axis orthogonal to the longitudinal axis “x” may correspond to a medial-lateral axis of the patient. In other configurations, the electrodes 32 may be disposed at any longitudinal or horizontal position along the distal portion 16 disposed between, proximal to, or distal to the defibrillation electrodes 28. In the example illustrated in FIG. 1A, electrodes 32 are disposed along the undulating configuration of distal portion 16 at locations that will be closer to heart 26 of patient 12 than defibrillation electrodes 28 (e.g., at a peak of the undulating configuration that is toward the left side of the sternum). As illustrated in FIG. 1A, for example, electrodes 32 are substantially aligned with one another along the left sternal line. In the example illustrated in FIG. 1A, defibrillation electrodes 28 are disposed along peaks of the undulating configuration that extend toward a right side of the sternum away from the heart. This configuration places pace/sense electrodes 32 at locations closer to the heart than electrodes 28, to facilitate cardiac pacing and sensing at relatively lower amplitudes.

In some examples, pace/sense electrodes 32 and the defibrillation electrodes 28 may be disposed in a common plane when distal portion 16 is implanted extravascularly. In other configurations, the undulating configuration may not be substantially disposed in a common plane. For example, distal portion 16 may define a concavity or a curvature.

Proximal end 14 of lead body 13 may include one or more connectors 34 to electrically couple lead 10 to ICD 9. ICD 9 may also include a connector assembly that includes electrical feedthroughs through which electrical connections are made between the one or more connectors 34 of lead 10 and the electronic components included within the housing. The housing of ICD 9 may house one or more processors, memories, transmitters, receivers, sensors, sensing circuitry, therapy circuitry, power sources (capacitors and batteries), and/or other components. The components of ICD 9 may generate and deliver electrical therapy such as anti-tachycardia pacing, cardioversion or defibrillation shocks, post-shock pacing, and/or bradycardia pacing.

The undulating configuration of distal portion 16 and the inclusion of electrodes 32 between defibrillation electrodes 28 provides a number of therapy vectors for the delivery of electrical therapy to the heart. For example, at least a portion of defibrillation electrodes 28 and one of electrodes 32 may be disposed over the right ventricle, or any chamber of the heart, such that pacing pulses and anti-tachyarrhythmia shocks may be delivered to the heart. The housing of ICD 9 may be charged with or function as a polarity different than the polarity of the one or more defibrillation electrodes 28 and/or electrodes 32 such that electrical energy may be delivered between the housing and the defibrillation electrode 28 and/or electrode 32 to the heart.

Each defibrillation electrode 28 may have the same polarity as every other defibrillation electrode 28 when a voltage is applied to it such that a shock may be delivered from all defibrillation electrodes together. In examples in which defibrillation electrodes 28 are electrically connected to a common conductor within lead body 13, this is the only configuration of defibrillation electrodes 28. However, in other examples, defibrillation electrodes 28 may be coupled to separate conductors within lead body 13 and may therefore each have different polarities such that electrical energy may flow between defibrillation electrodes 28, or between one of defibrillation electrodes 28 and one of pace/sense electrodes 32 or the housing electrode, to provide anti-tachyarrhythmia shock, pacing therapy, and/or to sense cardiac depolarizations. In this case, defibrillation electrodes 28 may still be electrically coupled together, e.g., via one or more switches within ICD 9, to have the same polarity.

In some examples, ICD system 8 may optionally include one or more shields 30. Shield 30 may be configured to be implanted in patient 12 separately from implantable lead 10 and/or distal portion 16 of lead 10, or with implantable lead 10 and/or distal portion 16. Shield 30 may have a variety of shapes, e.g., circular, elliptical, kidney bean shaped, balloon shaped, or the like. Shield 30 may be configured to impede an electric field from delivery of an electrical therapy via an electrode, e.g., from a pacing pulse and/or an anti-tachyarrhythmia shock, in a direction from the electrode away from the heart, e.g., in an anterior direction. In this manner, shield 30 may reduce the likelihood that the electrical field will stimulate extracardiac tissue, such as sensory or motor nerves. Furthermore, shield 30 may direct the electrical field toward the heart, allowing lower energy level pacing pulses to capture the heart and/or lower energy level anti-tachyarrhythmia shocks. than may be required without shield 30. Lower energy pacing pulses may also reduce the likelihood that pacing pulses delivered via the pace electrode or defibrillation pulses delivered via defibrillation electrodes 28 stimulate extracardiac tissue, and may result in less consumption of the power source of ICD 9 and, consequently, longer service life for the ICD. It should be understood that various aspects of the techniques of this disclosure may be applied to implantable systems other than ICD 9, including, but not limited to, bradycardia pacemaker systems. For example, a lead that does not include defibrillation electrodes 28 may include one or more shields 30 and may be used with a pacemaker system without defibrillation capabilities.

In the examples shown, shield 30 is implanted in the pleural cavity of patient 12, e.g., bounded laterally by pleurae 39, posteriorly by pericardium 38, and anteriorly by the sternum 22, and positioned anterior to distal portion 16. In some examples, shield 30 may be implanted such that at least a portion of shield 30 is position between the heart and lung (or lungs) of patient 12, e.g., between pericardium 38 and pleurae 39 of patient 12.

In some examples, distal portion 16 may be positioned in a substernal location via an introducer tool. For example, a clinician may create an incision 42 near a center of the torso of patient 12. The clinician may insert a dilator tool, e.g., a tunneling rod, into the lumen of the introducer tool, e.g., to structurally support the introducer tool during insertion of the introducer tool through tissue of patient 12. In some examples, a cross-sectional shape of the dilator tool may be similar to the cross-sectional shape of lumen of the introducer tool, e.g., circular, elliptical, square, rectangular, polygonal, or any suitable shape. In other examples, the introducer tool may comprise and/or be formed of a flexible or semi-flexible material, e.g., silicone, rubber, a polymer, polyvinyl chloride, latex, Teflon, or the like), and the cross-sectional shape of the dilator tool may be different from that of the lumen of the introducer tool. For examples, the dilator tool may have a circular cross-sectional shape to which the lumen of the introducer tool conforms when the dilator tool is within the lumen. In some examples, the entire cross-sectional shape of the introducer tool may conform to the cross-sectional shape of the dilator tool. The cross-sectional shape of the dilator tool, and the conforming introducer tool, may aid in inserting the introducer tool through tissue of patient 12, and/or aid in subsequently advancing distal portion 16 through the lumen of the introducer tool to the target positioning site, e.g., the target substernal location. For example, the clinician may insert the dilator tool within the lumen of the introducer tool, thereby altering the shape of the lumen and the introducer tool to the shape of the dilator tool, e.g., a circular cross-sectional shape. The clinician may then insert the introducer tool with the dilator tool within the lumen through tissue of the patient, accessed via the incision 42. The clinician may then remove the dilator tool, and the introducer tool and/or lumen of the introducer tool may relax to its preferred shape, e.g., elliptical, square, rectangular, polygonal, or the like. In other examples, the introducer tool and/or lumen does not conform to the shape of the dilator tool and may have the same and/or different cross-sectional shape as the dilator tool.

The clinician may insert the introducer tool with the dilator tool through tissue of patient 12 and into the substernal location via the incision 42, and advance the introducer tool within the substernal location from the incision 42 superior along a posterior of a sternum to form a substernal path 44. The clinician may then remove the dilator tool and insert distal portion 16 of medical lead 10 into the substernal location through the lumen of the introducer tool. The undulating configuration of distal portion 16 may be in a relatively straight configuration when being advanced through the lumen of the introducer tool. For example, the pre-formed or shaped undulating configuration of distal portion 16 is flexible enough to be straightened out while routing the lead through the lumen of the introducer tool (or sheath, or a channel, or the like). In some examples, the width of the lumen is smaller (e.g., narrower) than the width of the undulating configuration of distal portion 16. Once the distal portion is in place, the introducer tool is withdrawn toward the incision and removed from the body of the patient while leaving the lead in place along the substernal path. As the introducer tool is withdrawn, distal portion 16 takes on its pre-formed undulating configuration and, in examples in which shield 30 is included, shield 30 may transition to a deployed configuration.

In some examples, the cross-sectional shape of the lumen of the introducer tool may allow distal portion 16 to be at least partially in its undulating shape, thereby limiting rotational movement of distal portion 16 within the lumen. For example, when distal portion 16 is advanced through the lumen having a circular cross-sectional shape, distal portion 16 may be constrained by the lumen (e.g., by the width of the lumen) to be substantially straight. A circular cross sectional shape of the lumen may not provide resistance to rotational movement (e.g., other than friction of the inner walls defining the lumen) of distal portion 16, which may “corkscrew” and/or rotate while being advanced through the lumen. By contrast, the introducer tool has a cross-sectional shape configured to resist rotational movement of distal portion 16 while distal portion 16 is advanced through the lumen and/or when the introducer tool is removed from the patient to deploy distal portion 16, e.g., when the introducer tool is drawn back from medical lead 10 after distal portion 16 is positioned at the substernal target site. For example, the dilator tool may be configured to have a lumen having cross-sectional shape that has a non-rotationally symmetric inner extent and/or distance such that the inner walls of the lumen contact and resist rotational movement of distal portion 16 within the lumen, e.g., greater than the resistance (e.g., frictional) of a circularly cross-sectionally shaped lumen. The cross-sectional shape of the lumen may allow distal portion 16 to at least partially expand/deploy to at least a partial deployed/undulating configuration in a particular and/or predetermined rotational direction within the lumen, and the inner walls of the lumen may allow rotational movement of distal portion 16 within the lumen, e.g., in order to rotate within the lumen, distal portion 16 would need to straighten or the cross-sectional shape of the lumen would need to deform. In other words, the introducer tool may be configured to limit rotation of distal portion 16 within the lumen.

Once distal portion 16 is positioned at the target substernal location, the clinician may withdraw the introducer tool toward the incision 42 to remove the introducer tool from patient 12 while leaving medical lead 10 in place along substernal path 44. Distal portion 16 may then relax, expand, and/or other change to take its pre-formed undulating configuration, e.g., its deployed configuration, within the substernal location as it exits the introducer tool lumen. The clinician may insert and advance distal portion 16 with the pace/sense electrodes 32 to be disposed on the undulating configuration when deployed such that that undulating configuration pushes the pace/sense electrodes 32 toward the left side of the sternum compared to the defibrillation electrodes 28.

In some examples, the introducer tool provides the benefit of preventing and/or reducing “lead flips” in which the distal portion is delivered to the target substernal location and deploys to the undulating shape with the defibrillation electrodes 28 to the left side of patient 12 and pace/sense electrodes 32 to the right. For example, the introducer tool is configured to lock the rotational orientation of distal portion 16 to a predetermined rotational orientation during insertion of distal portion 16 such that distal portion 16 may deploy having the predetermined and/or a selected rotational orientation relative to the anatomy of patient 12, e.g., such that that undulating configuration extends the pace/sense electrodes 32 toward the left side of the sternum compared to the defibrillation electrodes 28.

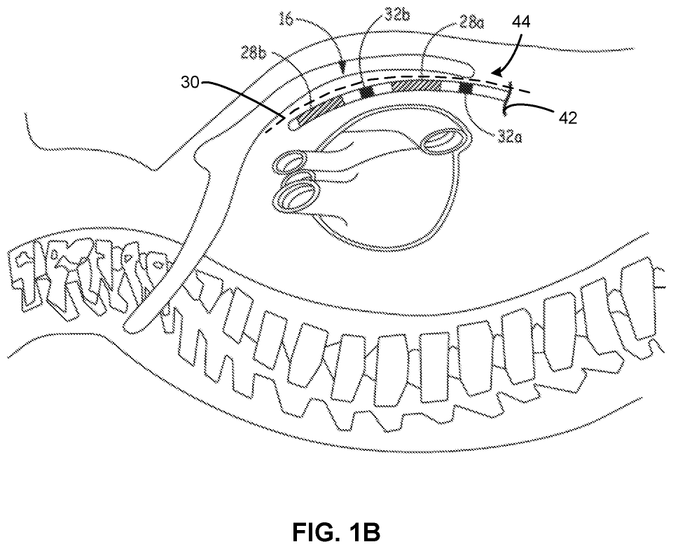

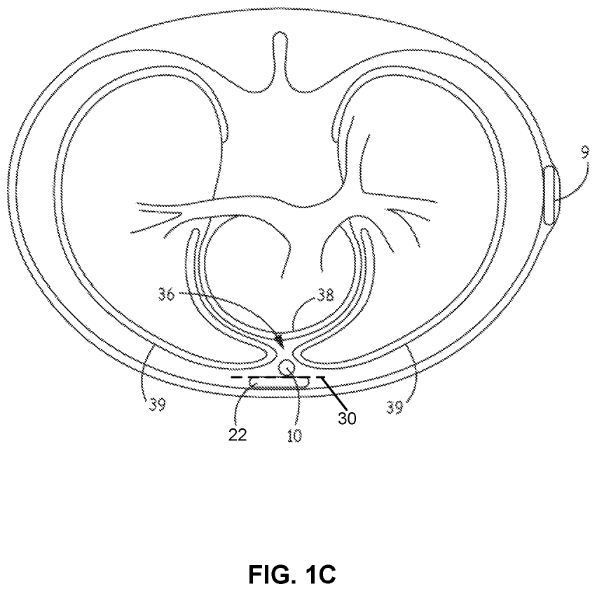

FIG. 2 is a conceptual diagram illustrating distal portion 16 of an example implantable medical lead 10 deployed in a first rotational orientation, and FIG. 3 is a conceptual diagram illustrating distal portion 16 of the example implantable medical lead 10 deployed in a second rotational orientation. In the example shown, distal portion 16 is illustrated as viewed from the perspective of an observer facing patient 12, e.g., viewing the anterior of patient 12. In the examples, shown the first rotational orientation of FIG. 2 may be a predetermined, “correct,” and/or “unflipped” rotational orientation in which the undulating configuration extends the pace/sense electrodes 32 toward the left side of sternal line A compared to defibrillation electrodes 28 (e.g., and towards the right of sternal line A from the perspective of the observer facing patient 12). The first rotational orientation of FIG. 3 may be an “incorrect,” and/or “flipped” rotational orientation in which the undulating configuration extends the pace/sense electrodes 32 toward the right side of sternal line A compared to defibrillation electrodes 28 (e.g., and towards the left of sternal line A from the perspective of the observer facing patient 12).

As illustrated in FIGS. 2 and 3, the undulating configuration of distal portion 16 may include a plurality of peaks along the length of the distal portion. In the example illustrated by FIG. 2, distal portion includes three peaks 24a, 24b, and 24c (collectively, “peaks 24”). Other configurations, however, may include any number of peaks 24.

The undulating configuration may define a peak-to-peak distance 35, which may be variable or constant along the length of distal portion 16. In the configuration illustrated in FIGS. 1-3, the undulating configuration defines a substantially sinusoidal configuration, with a constant peak-to-peak distance 35 of approximately 2.0-6.0 cm. The undulating configuration may also define a peak-to-peak width 37, which may also be variable or constant along the length of the undulating configuration. In the configuration illustrated in FIGS. 1-3, the undulating configuration defines a substantially sinusoidal shape, with a constant peak-to-peak width 37 of approximately 0.25-3.0 cm. However, in other instances, the undulating configuration may define other shapes and/or patterns, e.g., S-shapes, wave shapes, or the like.

Defibrillation electrodes 28 may extend along, e.g., be disposed on or cover, a substantial part of the undulating configuration of distal portion 16, e.g., along at least 80% of the undulating portion. Defibrillation electrodes 28 may extend along more or less than 80% of the undulating configuration. As another example, defibrillation electrodes 28 may extend along at least 90% of the undulating configuration.

Defibrillation electrode 28a extends along a substantial portion of the undulating configuration of distal portion 16 from the proximal end to peak 24b, e.g., along a substantial portion of the first “wave” associated with peak 24a, and the defibrillation electrode segment 28b extends along a substantial portion of the undulating configuration from peak 24b to the distal end of the undulating configuration, e.g., along a substantial portion of the second “wave” associated with peak 24c). In the example illustrated in FIGS. 1-3, a part of the undulating configuration on which defibrillation electrodes 28 are not disposed is a gap between defibrillation electrodes 28a and 28b, on peak 24b, where electrode 32b is disposed.

As illustrated in FIGS. 2 and 3, distal portion 16 of lead 10 may include lead body portion 40a and lead body portion 40b (collectively, “lead body portions 40”), and distal lead body portion 46. Lead body portions 40 extend between pace/sense electrode 32b and a respective one of defibrillation electrodes 28. Lead body portion 42 extends distally from defibrillation electrode 28b. Lead body portions 40 may provide a relatively even or smooth surface transition between the outer profile of pace/sense electrode 32b and the outer profiles of defibrillation electrodes 28. Conductors coupled to electrodes 32b and 28b may extend through lead body portion 40a, and a conductor coupled to electrode 28b may extend through lead body portion 40b. Lead body portions 40a and 40b, and 42, may be formed of one or more polymers, which may be the same as or different from shield 30, and/or other portions of lead body 13.

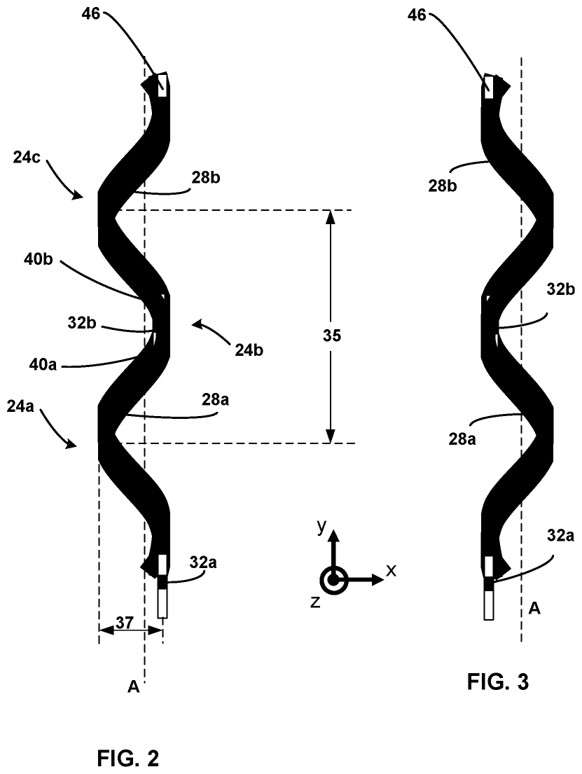

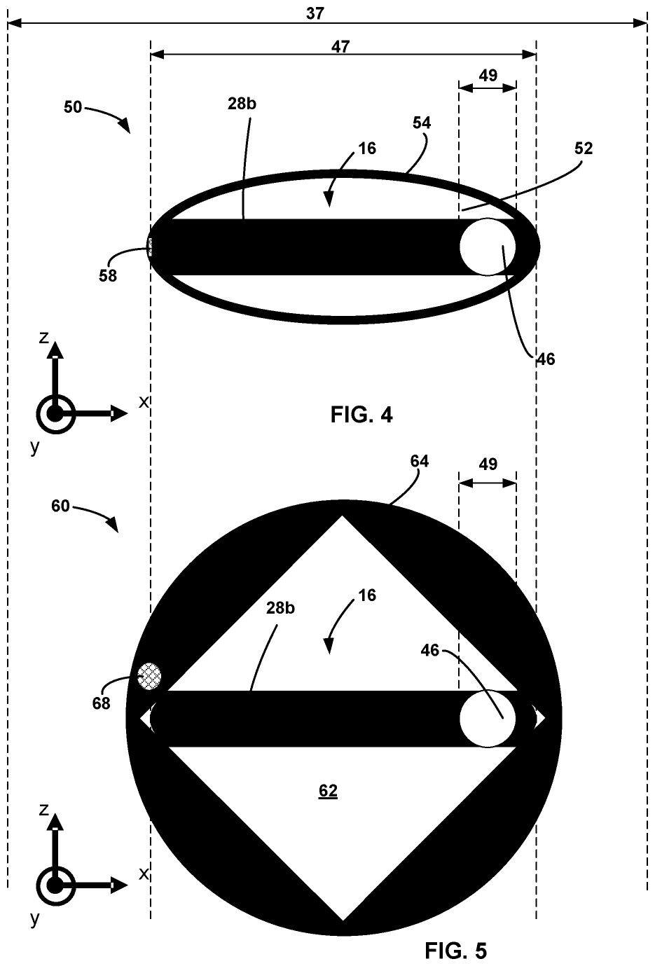

FIG. 4 is a conceptual, cross-sectional view of distal portion 16 of implantable medical lead 10 within a lumen 52 of an example introducer tool 50. FIG. 5 is a conceptual, cross-sectional view of a distal portion of an example implantable medical lead within a lumen 62 of another example introducer tool 60. Introducer tool 50 may comprise an elongated body 54 defining lumen 52 and configured to be inserted into patient 12, e.g., in some examples with a dilator tool, and in other examples without a dilator tool. Introducer tool 60 may comprise an elongated body 64 defining lumen 62 and configured to be inserted into patient 12, e.g., in some examples with a dilator tool, and in other examples without a dilator tool. Elongated bodies 54 and 64 are elongated in the y-direction as shown, e.g., having a major length in the y-direction, and the cross-sectional views of FIGS. 4 and 5 are at a length position at the distal end of elongated bodies 54 and 64, respectively. In some examples, FIGS. 4 and 5 may be a distal end view of elongated bodies 54 and 64, respectively.

In the example shown, introducer tool 50 has an elliptical cross-sectional shape. For example, elongated body 54 defines lumen 52 having an elliptical shape and an outer surface having an elliptical shape. Although both lumen 52 and the outer surface of elongated body 54 have the same shape (differing in size by the thickness of elongated body 54), in other examples the cross-sectional shape of lumen 52 and the outer surface of elongated body 54 may be different. For example, introducer tool 60 has a circular cross-sectional shape, e.g., the outer surface of elongated body 64 defines a circular cross section shape, and lumen 62 has a square cross-sectional shape, e.g., elongated body 64 defines a square cross-sectional shape. Although elongated body 54 defines lumen 52 with an elliptical cross-sectional shape and elongated body 64 defines lumen 62 with a square cross-sectional shape, in other examples, elongated body 54 and/or 64 may define a lumen having any other suitable cross-sectional shape, e.g., rectangular, polygonal, or any other cross-section shape having a non-circular and/or non-rotationally symmetric inner extent and/or distance (e.g., in the x-z plane as shown) such that the inner walls of lumen 52 and 62 contact and resist rotational movement of distal portion 16 within lumens 52 and 62.

In some examples, elongated body 54 and/or elongated body 64 may be a flexible material, for example, PVDF (polyvinylidene fluoride), nylon (polyamide), polyethylene terephthalate (PET), polytetrafluoroethylene (PTFE), high density polyethylene (HDPE), polycarbonate, silicone, a plastic, a polymer, or the like. In the examples shown, elongated bodies 54 and 64 define lumens 52 and 62, respectively, that are configured to receive at least distal portion 16 of medical lead 10 and that are configured to limit rotation of distal portion 16 within the respective lumens 52 and 62. For example, elongated bodies 54 and 64 may have a stiffness configured to straighten the undulating configuration of distal portion 16 upon insertion of distal portion 16 within lumen 52 or 62. In the examples shown, distal portion 16 is illustrated as received and/or inserted into lumens 52 and 62, respectively, with a constrained peak-to-peak width 47 within lumens 52 and 62 that is less than peak-to-peak width 37 of distal portion 16 when in the deployed, e.g., unconstrained, unstraightened configuration. In the examples shown, the cross-sectional shapes of lumens 52 and 62 are configured to allow distal portion 16 to at least partially expand, e.g., from a substantially straight configuration, to undulated in a particular rotational direction within lumens 52 and 62, e.g., the x-direction of the plurality of rotational directions in the x-z plane in the examples shown, thereby limiting rotational movement of distal portion 16. For example, if lumens 52 or 62 were to constrain distal portion 16 to be substantially straight, e.g., having a peak-to-peak width substantially the same as the diameter 49 of the tubular or cylindrical shape of distal portion 16 (and/or lead body 13), the force limiting rotational movement of distal portion 16 within lumens 52 and 62 may only be from friction between the inner surface of elongated bodies 54 and 64 and the outer surfaces of distal portion 16. In the examples shown, lumens 52 and 62 are configured to allow distal portion 16 to at least partially undulate, e.g., to constrain distal portion 16 to having a peak-to-peak width 47 that is greater than diameter 49, and in some examples less than fully expanded/deployed/undulating/unconstrained configuration peak-to-peak width 37. Additionally, the cross-sectional shapes of lumens 52 and 62 (e.g., elliptical and square, respectively) have differing extents and/or diameters as a functions of rotational direction, and the force limiting rotational movement of distal portion 16 is both from friction between the inner surface of elongated bodies 54 and 64 and the outer surfaces of distal portion 16 and a force required to either straighten distal portion 16 to allow distal portion 16 to rotationally move (e.g., elongated bodies 54 and/or 64 block rotational movement of distal portion 16 by decreasing the allowable widths of distal portion 16 in certain rotational directions) or a force required to deform elongated bodies 54 and 64 to allow distal portion 16 to rotationally move.

In some examples, introducer tool 50 and/or 60 may define lumens 52 and 62, respectively, having a different cross-sectional shape for a proximal portion of the lengths of introducer tool 50 and/or 60, e.g., circular (not shown), than the cross-sectional shape of lumen 52 and 62, respectively, for a distal portion of the lengths of introducer tool 50 and/or 60. For example, a clinician may form a path and/or tunnel through tissue of patient 12 via incision 42, and insert and advance distal portion 16 through lumen 52 or 62 from the proximal portion of the length of introducer tool 50 or 60, within which lumen 52 or 62 is configured to substantially straighten distal portion 16, to the distal portion of the length of introducer tool 50 or 60, within which lumen 52 or 62 is configured to allow distal portion 16 to at least partially expand to at least partially undulate and “lock” the rotational orientation of distal portion 16 relative to introducer tool 50 or 60. The clinician may then insert and advance introducer tool 50 or 60 to the target site while being able to control the rotational orientation of introducer tool 50 or 60 and know the rotational orientation of distal portion 16 and that the rotational orientation of distal portion 16 won't change as introducer tool 50 or 60 is advanced to the target substernal site/position. In some examples, whether introducer tool 50 or 60 is “pre-loaded” with distal portion 16 before insertion into patient 12 or inserted into patient 12 before inserting and advancing distal portion 16 through lumen 52 or 62, introducer tools 50 and 60 may include one or more radiopaque markers 58 and/or 68 configured to indicate a rotational orientation of introducer tool 50 and 60 within patient 12.

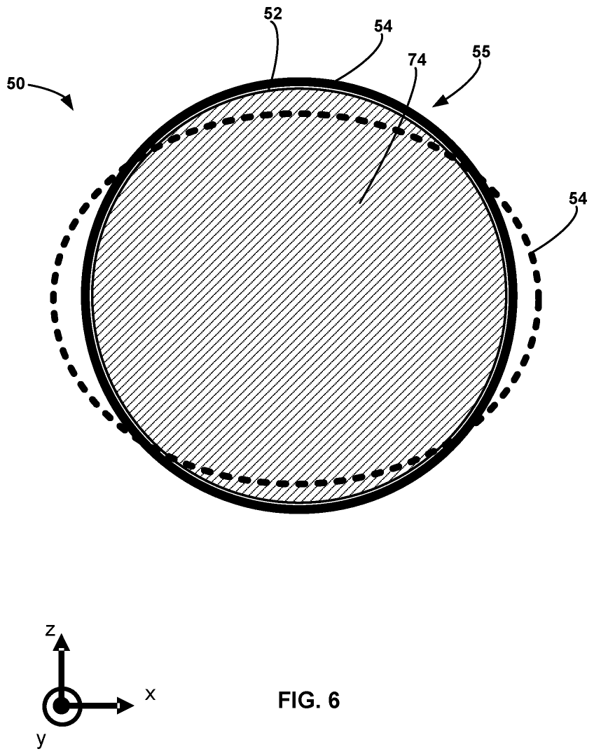

In some examples, a medical device and/or system includes ICD system 8, an introducer tool (e.g., introducer tool 50 and/or 60), and a dilator tool 74. FIG. 6 is a conceptual, cross-sectional view of an example dilator tool 74 within a lumen 62 of the example introducer tool 50 of FIG. 4. In some examples, dilator tool 74 may be used with introducer tool 50 or introducer tool 60, or any suitable introducer tool.

The dilator tool 74 may be configured to provide support and define a cross-sectional shape of introducer tool 50 and/or 60 or to “tunnel” through tissue of patient 12 with a cross-sectional shape, e.g., in preparation for inserting introducer tool 50 and/or 60. In the example shown in FIG. 6, introducer tool 50 may have an elliptical cross-sectional shape as described above before dilator tool 74 is inserted into lumen 52, e.g., illustrated as the dashed elliptical cross-sectional shape of elongated body 54 in FIG. 6. In some examples, dilator tool 74 is configured to be received within the lumen and configured to aid in inserting the introducer tool into a patient, or lumen 52 and elongated body 54, and/or lumen 62 and elongated body 64, are configured to receive the dilator within lumen 52 and/or lumen 62. For example, dilator tool 74 may comprise a material having a stiffness configured to support introducer tool 50 and/or 60 during insertion into patient 12 and through tissue of patient 12 to position introducer tool 50 and/or 60 to deliver distal portion 16 to a target location within patient 12, e.g., a substernal location. In some examples, dilator tool 74 may have a cross-sectional shape that matches the cross-sectional shape of lumen 52 or lumen 62, and is sized so as to be able to be received within lumen 52 and/or lumen 62, e.g., substantially the same as, or less than, the size of lumen 52 and/or lumen 62. In other examples, dilator tool 74 is configured to change the cross-sectional shape of lumen 52 and/or lumen 62 upon insertion of dilator tool 74 within lumen 52 or lumen 62. For example, elongated bodies 54 and/or 64 may comprise a flexible, compliant, and/or elastic material, and dilator tool 74 may have a greater stiffness and/or rigidity than elongated bodies 54 and/or 64 such that when inserted, the cross-sectional shape of lumen 52 or 62 at least partially conforms to that of dilator tool 74. In some examples, the cross-sectional shape of introducer tool 50 and/or 60, e.g., the cross-sectional shape of the outer surface of elongated body 54 and/or 64, may at least partially conform to that of dilator tool 74. For example, dilator tool 74 may be configured to reduce wounding and/or trauma to tissue of patient 12, e.g., shaped to reduce trauma to patient 12 during insertion of dilator tool 74 and/or introducer 50 or 60 into patient 12. For example, dilator tool 74 may be configured to change the cross-sectional shape of lumen 52 and the outer surface of elongated body 54 to have a circular cross-sectional shape 55 when inserted into lumen 52 (illustrated as elongated body 54 with the solid line in FIG. 6), and to provide support to elongated body 54 during insertion into and through tissue of patient 12. In some examples, dilator tool 74 may reduce a diameter of introducer tool 50, e.g., in the x-direction in the example shown, to reduce a cross-sectional area of the introducer/dilator tool inserted through tissue of patient 12.

In some examples, introducer tool 50 and/or 60 is configured to resume its shape after introducer tool 50 or 60 is inserted within patient 12 and after dilator tool 74 is removed from lumen 52 or 62, respectively. For example, elongated body 54 may be a flexible, compliant, and/or elastic material configured to resume the elliptical cross-sectional shape as shown in FIG. 4, and elongated body 64 may be a flexible, compliant, and/or elastic material configured to resume the circular cross-sectional shape of the outer surface of elongated body 64 and the square cross-sectional shape of lumen 62 as shown in FIG. 5.

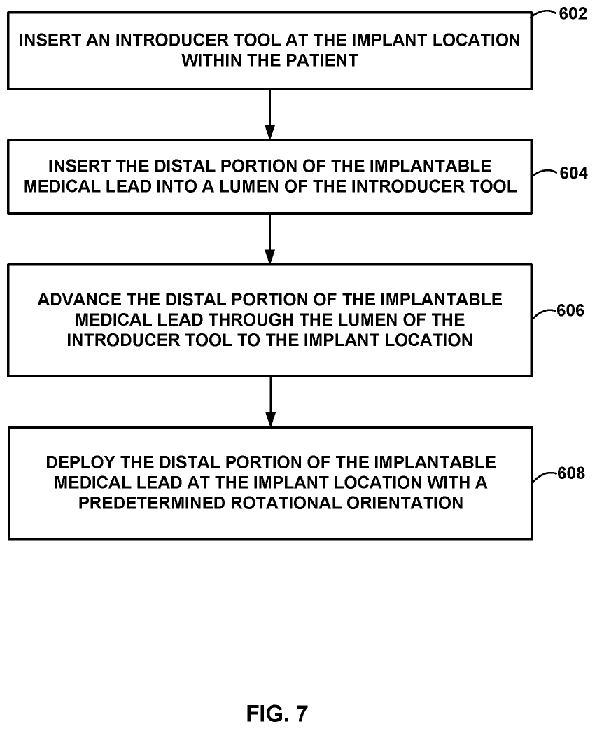

FIG. 7 is a flow diagram illustrating an example technique for implanting a medical device including an implantable medical lead 10. FIG. 7 is described with respect to implantable medical lead 10 and introducer tool 50. However, the example technique of FIG. 7 may be used to implant other leads, shields, or introducer tools, e.g., introducer tool 60.

A clinician may position and/or insert introducer tool 50 at an implant location within patient 12 (602). For example, the clinician may insert a dilator tool within lumen 52 of introducer tool 50. As described above, the dilator tool may be configured to change the cross-sectional shape of lumen 52, e.g., from elliptical to a substantially circular cross-sectional shape, and/or the dilator tool maybe configured to change the cross-sectional shape of introducer tool 50, e.g., the cross-sectional shape of the outer surface of elongated body 54, from elliptical to substantially circular, and elongated body 54 may be flexible, compliant, and/or elastic and configured to receive the dilator tool and change cross-sectional shape. In some examples, the clinician may then insert and/or position introducer tool 50 at the implant location within patient 12 with the aid of the dilator tool. The clinician may then remove, prior to inserting distal portion 16 of the implantable medical lead 10 into lumen 52, the dilator tool from lumen 52, and introducer tool 50 is configured to resume its shape (e.g., elliptical) after introducer tool 50 is inserted within patient 12 and the dilator tool is removed from lumen 52.

In other examples, introducer tool 50 may be inserted to the implant location within patient 12 without a dilator tool. For example, elongated body 54 may have a stiffness adequate for advancing introducer tool 50 through incision 42 and tissue of patient 12 to the implant location.

The clinician may insert distal portion 16 of implantable medical lead 10 into lumen 52 (604). For example, the clinician may insert distal portion 16 into an opening to lumen 52 at a proximal end of introducer tool 50, and introducer tool 52 may cause distal portion 16 to straighten, e.g., from its deployed, undulating configuration to a substantially straighter insertion configuration to be advanced through lumen 52 to the implant location.

The clinician may advance distal portion 16 through lumen 52 to the implant location (606). For example, the clinician may distally move distal portion 16 within lumen 52, and lumen 52 may limit rotational movement of distal portion 16 relative to lumen 52 and/or patient 12, e.g., to “lock” the rotational orientation of distal portion 16 to a predetermined rotational orientation. In some examples, the clinician may rotate introducer tool 50 to change the rotational orientation of both introducer tool 50 and distal portion 16 locked relative to lumen 52. In some examples, introducer tool 50 may include one or more radiopaque markers configured to indicate the rotational orientation of introducer tool 50 and/or distal portion 16, and the clinician may rotate introducer tool 50 based on the position of the one or more radiopaque markers prior to deploying distal portion 16 in the undulating configuration, e.g., such that the undulating configuration of distal portion 16 is at a predetermined rotational orientation when deployed and/or exits lumen 52.

The clinician may deploy distal portion 16 to be in the undulating configuration at the implant location with the predetermined rotational orientation (608). For example, the clinician may draw back and/or move introducer tool 50 in the proximal direction to release and/or cause distal portion 16 to exit lumen 52 at the implant location and to deploy, upon exiting lumen 52, to the undulating configuration oriented in the predetermined rotational direction, e.g., with defibrillation electrodes 28 oriented towards the patient's right side, and pacing electrodes 32 towards the patient's left side.

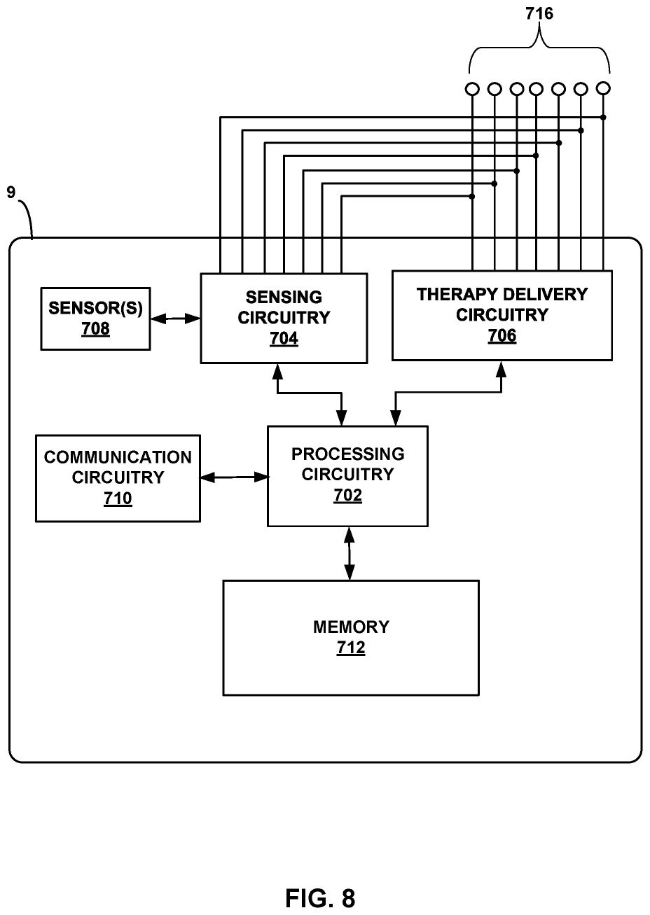

FIG. 8 is a functional block diagram of an example configuration of electronic components and other components of ICD 9. ICD 9 includes a processing circuitry 702, sensing circuitry 704, therapy delivery circuitry 706, sensors 708, communication circuitry 710, and memory 712. In other examples, ICD 9 may include more or fewer components. The described circuitry and other components may be implemented together on a common hardware component or separately as discrete but interoperable hardware or software components. Depiction of different features is intended to highlight different functional aspects and does not necessarily imply that such circuitry and other components must be realized by separate hardware or software components. Rather, functionality associated with one or more circuitries and components may be performed by separate hardware or software components, or integrated within common or separate hardware or software components.

Sensing circuitry 704 may be electrically coupled to some or all of electrodes 716, which may correspond to any of the defibrillation, pace/sense, and housing electrodes described herein. Sensing circuitry 704 is configured to obtain signals sensed via one or more combinations of electrodes 716 and process the obtained signals.

The components of sensing circuitry 704 may be analog components, digital components or a combination thereof. Sensing circuitry 704 may, for example, include one or more sense amplifiers, filters, rectifiers, threshold detectors, analog-to-digital converters (ADCs) or the like. Sensing circuitry 704 may convert the sensed signals to digital form and provide the digital signals to processing circuitry 702 for processing or analysis. For example, sensing circuitry 704 may amplify signals from the sensing electrodes and convert the amplified signals to multi-bit digital signals by an ADC. Sensing circuitry 704 may also compare processed signals to a threshold to detect the existence of atrial or ventricular depolarizations (e.g., P- or R waves) and indicate the existence of the atrial depolarization (e.g., P-waves) or ventricular depolarizations (e.g., R-waves) to processing circuitry 702. As shown in FIG. 8, ICD 9 may additionally include one or more sensors 708, such as one or more accelerometers, which may be configured to provide signals indicative of other parameters of a patient, such as activity or posture, to processing circuitry 702.

Processing circuitry 702 may process the signals from sensing circuitry 704 to monitor electrical activity of heart 26 of patient 12. Processing circuitry 702 may store signals obtained by sensing circuitry 704 as well as any generated EGM waveforms, marker channel data or other data derived based on the sensed signals in memory 712. Processing circuitry 702 may analyze the EGM waveforms and/or marker channel data to detect arrhythmias (e.g., bradycardia or tachycardia). In response to detecting the cardiac event, processing circuitry 702 may control therapy delivery circuitry 706 to deliver the desired therapy to treat the cardiac event, e.g., defibrillation shock, cardioversion shock, ATP, post shock pacing, or bradycardia pacing.

Therapy delivery circuitry 706 is configured to generate and deliver electrical therapy to heart 26. Therapy delivery circuitry 706 may include one or more pulse generators, capacitors, and/or other components capable of generating and/or storing energy to deliver as pacing therapy, defibrillation therapy, cardioversion therapy, cardiac resynchronization therapy, other therapy or a combination of therapies. In some instances, therapy delivery circuitry 706 may include a first set of components configured to provide pacing therapy and a second set of components configured to provide defibrillation therapy. In other instances, therapy delivery circuitry 706 may utilize the same set of components to provide both pacing and defibrillation therapy. In still other instances, therapy delivery circuitry 706 may share some of the defibrillation and pacing therapy components while using other components solely for defibrillation or pacing. Processing circuitry 702 may control therapy delivery circuitry 706 to deliver the generated therapy to heart 26 via one or more combinations of electrodes 716. Although not shown in FIG. 8, ICD 9 may include switching circuitry configurable by processing circuitry 702 to control which of electrodes 716 is connected to therapy delivery circuitry 706 and sensing circuitry 704.

Communication circuitry 710 includes any suitable hardware, firmware, software or any combination thereof for communicating with another device, such as a clinician programmer, a patient monitoring device, or the like. For example, communication circuitry 710 may include appropriate modulation, demodulation, frequency conversion, filtering, and amplifier components for transmission and reception of data with the aid of an antenna.

The various components of ICD 9 may include any one or more processors, controllers, digital signal processors (DSPs), application specific integrated circuits (ASICs), field-programmable gate arrays (FPGAs), or equivalent discrete or integrated circuitry, including analog circuitry, digital circuitry, or logic circuitry. Processing circuitry 702 may include fixed function circuitry and/or programmable processing circuitry. The functions attributed to processing circuitry 702 herein may be embodied as software, firmware, hardware or any combination thereof.

Memory 712 may include computer-readable instructions that, when executed by processing circuitry 702 or other components of ICD 9, cause one or more components of ICD 9 to perform various functions attributed to those components in this disclosure. Memory 712 may include any volatile, non-volatile, magnetic, optical, or electrical media, such as a random-access memory (RAM), read-only memory (ROM), non-volatile RAM (NVRAM), static non-volatile RAM (SRAM), electrically-erasable programmable ROM (EEPROM), flash memory, or any other non-transitory computer-readable storage media.

In some examples, rather than extending in a superior direction along the sternum, the distal portion of the lead may be oriented orthogonal or otherwise transverse to the sternum and/or inferior to the heart. In such examples, the lead may include one or more shields that cover a portion of an outer surface of one or more electrodes, e.g., an anterior and/or inferior portion, according to any of the examples described herein. Such shield(s) may impede an electrical field in a direction away from the heart, which may be an anterior and/or inferior direction.

It will be appreciated by persons skilled in the art that the present application is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the application, which is limited only by the following claims.

The following examples are a non-limiting list of clauses in accordance with one or more techniques of this disclosure.

Example 1. A medical device system comprising: an implantable medical lead comprising: a lead body defining a proximal end and a distal portion, wherein at least a part of the distal portion of the lead body defines an undulating configuration; and an introducer tool defining a lumen configured to receive the distal portion, wherein the introducer tool is configured to limit rotation of the distal portion within the lumen.

Example 2. The medical device system of Example 1, wherein the lumen has a cross-sectional shape that is at least one of rectangular, square, or elliptical.

Example 3. The medical device system of Example 2, wherein the lumen has a circular cross-sectional shape at the proximal end of the introducer tool and the at least one of rectangular, square, or elliptical cross-sectional shape at the distal portion of the introducer tool.

Example 4. The medical device system of any one of Examples 1-3, wherein the cross-sectional shape of the lumen is configured to limit rotation of the distal portion within the lumen.

Example 5. The medical device system of any one of Examples 1-4, wherein the introducer tool is configured to straighten the undulating configuration of the distal portion upon insertion of the distal portion within the lumen, wherein the cross-sectional shape of the lumen is configured to allow the distal portion to at least partially expand to undulate in a particular rotational direction within the lumen thereby limiting rotational movement of the distal portion, wherein a first width of the lumen is smaller than a second width of the distal portion of the lead body.

Example 6. The medical device system of any one of Examples 1-5, further comprising a dilator tool configured to be received within the lumen and configured to aid in inserting the introducer tool into a patient.

Example 7. The medical device system of Example 6, wherein the introducer tool comprises a flexible material, wherein the dilator tool is configured to change the cross-sectional shape of the lumen upon insertion of the dilator tool within the lumen.

Example 8. The medical device system of any one of Examples 6 or 7, wherein the introducer tool is configured to resume its shape after the introducer tool is inserted within the patient and the dilator tool is removed from the lumen.

Example 9. The medical device system of any one of Examples 1-8, wherein the introducer tool comprises a radiopaque marker configured to indicate a rotational orientation of the introducer tool within a patient.

Example 10. The medical device system of any one of Examples 1-9, wherein the implantable medical lead further comprises: a first defibrillation electrode and a second defibrillation electrode disposed along the undulating configuration, the first and second defibrillation electrodes configured to deliver anti-tachyarrhythmia shocks; and a pace electrode configured to deliver a pacing pulse that generates an electric field proximate to the pace electrode.

Example 11. A method comprising: positioning an introducer tool at an implant location within a patient, wherein the introducer tool defines a lumen configured to receive a distal portion of an implantable medical lead, wherein the introducer tool is configured to limit rotation of the distal portion within the lumen; inserting the distal portion of the implantable medical lead into the lumen, wherein the implantable medical lead comprises a lead body defining a proximal end and the distal portion, wherein at least a part of the distal portion of the lead body defines an undulating configuration; advancing, through the lumen, the distal portion to the implant location; and deploying the distal portion to be in the undulating configuration at the implant location and with a predetermined rotational orientation.

Example 12. The method of Example 11, further comprising: rotating, prior to deploying the distal portion in the undulating configuration at the implant location, the introducer tool such that the undulating configuration is at the predetermined rotational orientation

Example 13. The method of any one of Example 11 or Example 12, wherein the lumen has a cross-sectional shape that is at least one of rectangular, square, or elliptical.

Example 14. The method of any one of Examples 11-13, wherein the cross-sectional shape of the lumen is configured to limit rotation of the distal portion within the lumen.

Example 15. The method of any one of Examples 11-14, wherein the introducer tool is configured to straighten the undulating configuration of the distal portion upon insertion of the distal portion within the lumen, wherein a cross-sectional shape of the lumen is configured to allow the undulating configuration to at least partially expand to undulate in a particular rotational direction within the lumen thereby limiting rotational movement of the undulating configuration.

Example 16. The method of any one of Examples 11-15, wherein the introducer tool comprises a flexible material, the method further comprising: inserting the dilator tool within the lumen, wherein inserting the dilator tool within the lumen changes the shape of the lumen to a substantially circular cross-sectional shape, wherein the introducer tool is positioned at the implant location within the patient with the aid of the dilator tool.

Example 17. The method of any one of Examples 11-16, further comprising: removing, prior to inserting the distal portion of the implantable medical lead into the lumen, the dilator tool from the lumen, wherein the introducer tool is configured to resume its shape after the introducer tool is inserted within the patient and the dilator tool is removed from the lumen.

Example 18. The method of any one of Examples 11-17, wherein the introducer tool comprises a radiopaque marker configured to indicate a rotational orientation of the introducer tool within a patient.

Example 19. A medical device system comprising: an implantable medical lead comprising a lead body defining a proximal end and a distal portion, wherein at least a part of the distal portion of the lead body defines an undulating configuration; and an introducer tool defining a non-circular lumen configured to receive the undulating configuration and to lock the undulating configuration to a particular rotational orientation within the lumen.

Example 20. The medical device system of Example 19, wherein the non-circular lumen has a cross-sectional shape that is at least one of rectangular, square, or elliptical.

Example 21. The medical device system of any one of Example 19 or Example 20, wherein the introducer tool comprises a flexible material, the medical device system further comprising a dilator tool configured to be received within the lumen and configured to change the cross-sectional shape of the lumen to a circular cross-sectional shape configured to reduce displacement of tissue of the patient during insertion of the introducer tool.

Example 22. The medical device system of any one of Examples 19-21, wherein the introducer tool comprises a radiopaque marker configured to indicate a rotational orientation of the introducer tool within a patient.

1. A medical device system comprising:
an implantable medical lead comprising a lead body defining a proximal end and a distal portion, wherein at least a part of the distal portion of the lead body defines an undulating configuration; and
an introducer tool defining a lumen configured to receive the distal portion, wherein the introducer tool is configured to limit rotation of the distal portion within the lumen.

2. The medical device system of claim 1, wherein the lumen has a cross-sectional shape that is at least one of rectangular, square, or elliptical.

3. The medical device system of claim 2, wherein the lumen has a circular cross-sectional shape at the proximal end of the introducer tool and the at least one of rectangular, square, or elliptical cross-sectional shape at the distal portion of the introducer tool.

4. The medical device system of claim 1, wherein the cross-sectional shape of the lumen is configured to limit rotation of the distal portion within the lumen.

5. The medical device system of claim 4, wherein the introducer tool is configured to straighten the undulating configuration of the distal portion upon insertion of the distal portion within the lumen, wherein the cross-sectional shape of the lumen is configured to allow the distal portion to at least partially expand to undulate in a particular rotational direction within the lumen thereby limiting rotational movement of the distal portion, wherein a first width of the lumen is smaller than a second width of the distal portion of the lead body.

6. The medical device system of claim 1, further comprising a dilator tool configured to be received within the lumen and configured to aid in inserting the introducer tool into a patient.

7. The medical device system of claim 6, wherein the introducer tool comprises a flexible material, wherein the dilator tool is configured to change the cross-sectional shape of the lumen upon insertion of the dilator tool within the lumen.

8. The medical device system of claim 7, wherein the introducer tool is configured to resume its shape after the introducer tool is inserted within the patient and the dilator tool is removed from the lumen.

9. The medical device system of claim 1, wherein the introducer tool comprises a radiopaque marker configured to indicate a rotational orientation of the introducer tool within a patient.

10. The medical device system of claim 1, wherein the implantable medical lead further comprises:
a first defibrillation electrode and a second defibrillation electrode disposed along the undulating configuration, the first and second defibrillation electrodes configured to deliver anti-tachyarrhythmia shocks; and a pace electrode configured to deliver a pacing pulse that generates an electric field proximate to the pace electrode.

11. A method comprising:
positioning an introducer tool at an implant location within a patient, wherein the introducer tool defines a lumen configured to receive a distal portion of an implantable medical lead, wherein the introducer tool is configured to limit rotation of the distal portion within the lumen;
inserting the distal portion of the implantable medical lead into the lumen, wherein the implantable medical lead comprises a lead body defining a proximal end and the distal portion, wherein at least a part of the distal portion of the lead body defines an undulating configuration;
advancing, through the lumen, the distal portion to the implant location; and
deploying the distal portion to be in the undulating configuration at the implant location and with a predetermined rotational orientation.

12. The method of claim 11, further comprising:
rotating, prior to deploying the distal portion in the undulating configuration at the implant location, the introducer tool such that the undulating configuration is at the predetermined rotational orientation.

13. The method of claim 11, wherein the lumen has a cross-sectional shape that is at least one of rectangular, square, or elliptical.

14. The method of claim 11, wherein the cross-sectional shape of the lumen is configured to limit rotation of the distal portion within the lumen.

15. The method of claim 11, wherein the introducer tool is configured to straighten the undulating configuration of the distal portion upon insertion of the distal portion within the lumen, wherein a cross-sectional shape of the lumen is configured to allow the undulating configuration to at least partially expand to undulate in a particular rotational direction within the lumen thereby limiting rotational movement of the undulating configuration.

16. The method of claim 11, wherein the introducer tool comprises a flexible material, the method further comprising:
inserting the dilator tool within the lumen, wherein inserting the dilator tool within the lumen changes the shape of the lumen to a substantially circular cross-sectional shape, wherein the introducer tool is positioned at the implant location within the patient with the aid of the dilator tool.

17. The method of claim 16, further comprising:
removing, prior to inserting the distal portion of the implantable medical lead into the lumen, the dilator tool from the lumen, wherein the introducer tool is configured to resume its shape after the introducer tool is inserted within the patient and the dilator tool is removed from the lumen.

18. The method of claim 11, wherein the introducer tool comprises a radiopaque marker configured to indicate a rotational orientation of the introducer tool within a patient.

19. A medical device system comprising:
an implantable medical lead comprising a lead body defining a proximal end and a distal portion, wherein at least a part of the distal portion of the lead body defines an undulating configuration; and
an introducer tool defining a non-circular lumen configured to receive the undulating configuration and to lock the undulating configuration to a particular rotational orientation within the lumen.

20. The medical device system of claim 19, wherein the non-circular lumen has a cross-sectional shape that is at least one of rectangular, square, or elliptical.

21. The medical device system of claim 19, wherein the introducer tool comprises a flexible material, the medical device system further comprising a dilator tool configured to be received within the lumen and configured to change the cross-sectional shape of the lumen to a circular cross-sectional shape configured to reduce displacement of tissue of the patient during insertion of the introducer tool.

22. The medical device system of claim 19, wherein the introducer tool comprises a radiopaque marker configured to indicate a rotational orientation of the introducer tool within a patient.
